(19) United States
(12) Patent Application Publication
(10) Pub. No.: US 2020/0197580 A1
(43) Pub. Date: Jun. 25, 2020

(54) IN-LINE WOUND FLUID SAMPLING SYSTEMS AND METHODS FOR USE WITH NEGATIVE PRESSURE WOUND THERAPY

(71) Applicant: KCI Licensing, Inc., San Antonio, TX (US)

(72) Inventors: Deepak V. KILPADI, San Antonio, TX (US); Christopher Allen CARROLL, San Antonio, TX (US)

(21) Appl. No.: 16/638,999

(22) PCT Filed: Aug. 14, 2018
(86) PCT No.: PCT/US2018/046758

§ 371 Date: Feb. 13, 2020

Related U.S. Application Data
(60) Provisional application No. 62/548,807, filed on Aug. 22, 2017.

(51) Int. Cl. A61M 1/00

(52) U.S. Cl. CPC A61M 1/0082; A61M 1/009; A61M 2205/3303; A61M 2205/3324; A61F 13/00068; A61M 2205/583; A61M 2205/3337; A61M 2205/3389; A61M 2205/3368

(57) ABSTRACT

Some embodiments provide in-line negative pressure wound therapy (NPWT) sampling assessment systems, comprising: a housing; a wound fluid input port configured to fluidly couple with a first wound fluid lumen extending from a wound treatment site; a wound fluid output port configured to fluidly couple with a NPWT control unit; an in-line sampling chamber positioned within the housing and comprising a theranostic sampling element, wherein the first in-line sampling chamber is configured to: removably and fluidly couple between the input port and the output port in-line with a wound fluid path between the wound treatment site and the NPWT control unit with the first theranostic sampling element positioned so that at least some of the wound fluid contacts at least a portion of the first theranostic sampling element; and decouple out of the wound fluid path without interrupting a negative pressure treatment process applied at the wound treatment site.

### TECHNICAL FIELD

The present invention relates generally to medical treatment systems, and more particularly, but not by way of limitation, to reduced-pressure wound therapy or treatment systems.

### BACKGROUND

Clinical studies and practice have shown that providing a reduced pressure in proximity to a wound site augments and accelerates the growth of new tissue at the wound site. The applications of this phenomenon are numerous, but application of reduced pressure has been particularly successful in treating wounds. This treatment (frequently referred to in the medical community as negative pressure wound therapy (NPWT), “negative pressure therapy” “reduced pressure therapy,” or “vacuum therapy”) provides a number of benefits, which may include faster healing and increased formulation of granulation tissue.

Negative pressure therapy, or reduced-pressure therapy, has been used to promote healing across a wide range of wound types. Typically, a manifold is inserted into the wound, and a drape is used to cover and seal the wound. The sealed area is fluidly coupled to a reduced-pressure unit to provide negative pressure, or reduced pressure, to the wound site. While this approach has produced meaningful results, shortcomings and areas of desired of improvement remain.

### BRIEF DESCRIPTION OF THE DRAWINGS

The above needs are at least partially met through apparatuses, systems and methods to provide in-line therapy theranostic sampling and/or diagnostics described in the following detailed description, particularly when studied in conjunction with the drawings, wherein:

FIG. 1 shows a simplified block diagram of an exemplary treatment system to treat a wound site on a subject/patient with reduced pressure, in accordance with some embodiments;

FIG. 2 illustrates a simplified block diagram of an exemplary in-line sampling assessment system with a rotational chamber switching system, in accordance with some embodiments;

FIG. 3 illustrates a simplified block diagram, partially transparent view of an exemplary in-line sampling assessment system with a linear chamber switching system, in accordance with some embodiments;

FIG. 4 illustrates a simplified block diagram, partially transparent view of an exemplary in-line sampling assessment system, in accordance with some embodiments;

FIG. 5 illustrates a simplified cross-sectional view of a portion of an exemplary input port, in accordance with some embodiments;

FIG. 6 illustrates a simplified plane view of an exemplary extraction tube, in accordance with some embodiments;

FIG. 7 illustrates a simplified flow diagram of an exemplary process of implementing a wound treatment, in accordance with some embodiments;

FIG. 8 illustrates a simplified flow diagram of an exemplary process of implementing a treatment therapy, in accordance with some embodiments; and

FIG. 9 illustrates an exemplary system for use in implementing methods, techniques, devices, apparatuses, systems, servers, sources and the like in providing user interactive virtual environments in accordance with some embodiments.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein.

### DETAILED DESCRIPTION

The following description is not to be taken in a limiting sense, but is made merely for the purpose of describing the general principles of exemplary embodiments. The scope of the invention should be determined with reference to the claims. Further, in the following descriptions, reference is made to the accompanying drawings that form a part hereof, and in which is shown examples embodiments. It is understood that modifications, changes and other embodiments may be utilized and that logical structural, mechanical, electrical, and chemical changes may be made without departing from the spirit or scope of the invention. Reference throughout this specification to “one embodiment,” “an embodiment,” “some embodiments”, “an implementation”, “some implementations”, “some applications”, or similar language means that a particular feature, structure, or characteristic described in connection with the embodiment is included in at least one embodiment of the present invention. Thus, appearances of the phrases “in one embodiment,” “in an embodiment,” “in some embodiments”, “in some implementations”, and similar language throughout this specification may, but do not necessarily, all refer to the same embodiment.

Some embodiments described herein provide systems, methods, processes and apparatuses to implement, manage and/or assist in implementing a wound treatment. Further, in some implementations, the systems, methods, processes and/or apparatuses implement, manage and/or assist in the management of reduced pressure delivered to a tissue site. Reduced pressure generally refers to a pressure less than the ambient pressure at a tissue site that is being subjected to treatment. In most cases, this reduced pressure will be less than the atmospheric pressure of the location at which the patient is located. Although the terms “vacuum” and “negative pressure” may be used to describe the pressure applied to the tissue site, the actual pressure applied to the tissue site may be significantly less than the pressure normally associated with a complete vacuum. Consistent with this nomenclature, an increase in reduced pressure or vacuum pressure refers to a relative reduction of absolute pressure, while a decrease in reduced pressure or vacuum pressure refers to a relative increase of absolute pressure. In some implementations, the system includes a reduced pressure source that generates reduced pressure. A reduced pressure source can be a device capable of generating reduced pressure and that can be fluidly cooperated to the treatment site such that the reduced pressure is applied at the site. In some embodiments, the reduced pressure source comprises a motor and pump, and the motor drives the pump to generate the reduced pressure. The reduced pressure may be delivered to the tissue site via a delivery tube.

Some embodiments described herein provide systems, methods, processes and apparatuses to implement, manage and/or assist in implementing a wound treatment. Further, in some implementations, the systems, methods, processes and/or apparatuses implement, manage and/or assist in the management of reduced pressure delivered to a tissue site. Reduced pressure generally refers to a pressure less than the ambient pressure at a tissue

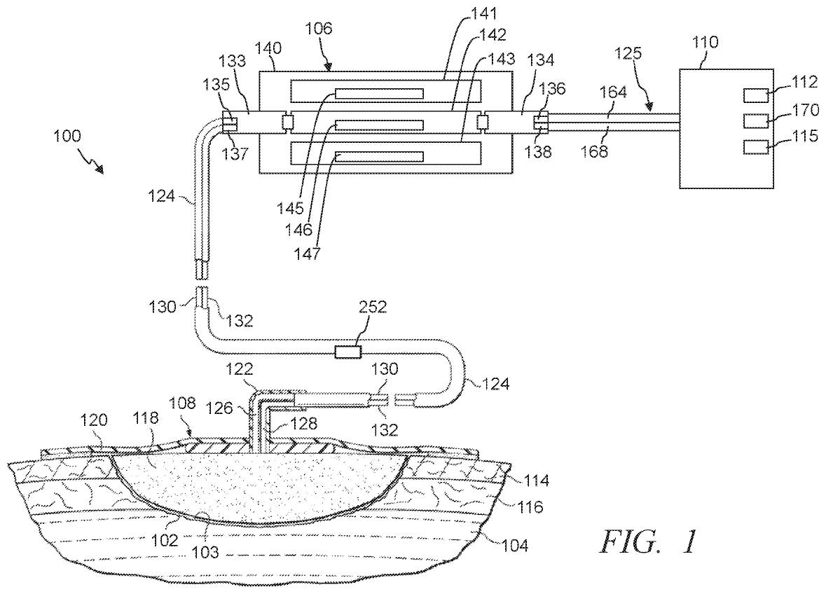

FIG. 1 is simplified block diagram of an exemplary treatment system 100 to treat a wound site on a subject/patient 104 (e.g., animal, human, harvest tissue, etc.), with reduced pressure is presented, in accordance with some embodiments. The treatment system 100 includes an in-line sampling assessment system 106, in some applications is implemented as a NPWT sampling assessment system. The treatment system 100 is shown on a human, but the treatment system 100 may be used on any animal 104, e.g., horse, cow, dog, pig, turtle, etc. (generally referred to as patient). Further, the treatment system 100 in some embodiments includes one or more therapy units 110 or other control circuitry, a reduced pressure source 112, which may be part of or separate from the therapy unit 110, and couples with and/or includes a wound dressing 108 (or other fluid reception device). Wound fluids (gas and/or liquid) may be extracted from the wound, and at least a portion of that wound fluid can be directed to the sampling assessment system 106. In some implementations, some of the wound fluid is additionally or alternatively communicated to a wound fluid collection unit 170. The wound fluid(s) are removed from the wound site using reduced pressure. Further, the wound fluid(s) may include fluids and/or gases emitted from the wound 103 itself (sometimes referred to as exudate), and in some instances may additionally or alternatively include fluids applied to an exemplary wound treatment site 102 as part of a treatment protocol (e.g., water, saline, debriding chemical (e.g., chemical suitable for softening, digesting, partially digesting, breaking down or partially breaking down undesirable tissue at the tissue site, which may include, without limitation, collagenase, ficin, pepsin, trypsin, chymotrypsin, papain, elastase, enzymes isolated from any suitable insect larvae, one or more analogues thereof, etc.), flushing agent, medications, antibacterials, growth factors, and various solutions.

The therapy unit 110 and/or control circuity may be utilized to provide control over a treatment protocol and/or pressure applied to the wound site. In controlling the protocol, the therapy unit may control whether, when and at what pressure a negative pressure is applied at a treatment site, control an application and/or extraction of one or more fluids from the treatment site, direct the application of one or more medications, directing the re-dressing of a wound site, generate one or more notifications, alarms or the like, recording and/or tracking sensor data and/or conditions of the treatment protocol, activating and/or controlling the sampling assessment system 106, directing a modification of the sampling assessment system 106, and other such aspects of a treatment protocol. In some embodiments, the therapy unit includes one or more control circuits configured to process data, such as data from a negative pressure sensor system 115, the sampling assessment system 106, and the like. A therapy unit may control the operation of one or more components of the treatment system 100. In some implementations, the therapy unit 110 and/or the reduced pressure source 112 directs and/or controls the motor of the reduced pressure source 112 (e.g., monitors the voltage across the motor to ascertain and control the supply pressure generated by the pump). For example, the therapy unit 110 may be implemented through a V.A.C.ULTA™ Therapy System, ACTIV.A.C.™ Therapy System, INFOV.A.C.™ Therapy System, V.A.C. FREEDOM™ Therapy System, V.A.C. SIMPLICITY™ Negative Pressure Wound Therapy System, V.A.C.VIA™ Therapy System, from KCI of San Antonio, Tex.; and other such therapy systems.

In some embodiments, one or more site extraction tubes 124 or conduits fluidly couple the treatment site 102 and the sampling assessment system 106. In some implementations, the site extraction tube 124 may be a multi-lumen tube or conduit. Similarly, one or more unit extraction tubes 125 or conduits fluidly coupling the sampling assessment system 106 and the therapy unit 110 and/or reduced pressure source 112. Again, one or more of the unit extraction tubes may be a multi-lumen tube or conduit.

The treatment system 100 may allow the user to position the weight of the in-line sampling assessment system 106 and the therapy unit 110 at different locations relative to and/or on the patient. In other words, the weight of the components of the treatment system 100 may be distributed at different locations on or around the patient. For example, one or more in-line sampling assessment systems 106 may be strapped to a portion of the patient 104, such as a leg, using straps or other attachment devices. At the same time, the therapy unit 110 may be attached at another location on the patient 104, e.g., a torso, using straps or other attachment devices. In other instances, one or both of the in-line sampling assessment system 106 and therapy unit 110 may be on a table, rack, wheeled cart, or other such device.

The wound 103 at the wound treatment site 102 may be the bodily tissue of any human, animal, or other organism, including bone tissue, adipose tissue, muscle tissue, dermal tissue, vascular tissue, connective tissue, cartilage, tendons, ligaments, or any other tissue. The reduced-pressure wound treatment system 100 is shown on an exemplary wound, which may for example be wound extending through and/or into an epidermis (or generally skin), the dermis, a hypodermis, subcutaneous tissue, etc.

A wound 103 at the wound treatment site 102 may be the bodily tissue of any human, animal, or other organism, including bone tissue, adipose tissue, muscle tissue, dermal tissue, vascular tissue, connective tissue, cartilage, tendons, ligaments, or any other tissue. The reduced-pressure wound treatment system 100 is shown cooperated with the exemplary wound treatment site 102, which may for example be one or more wounds extending through and/or into an epidermis 114 (or generally skin), the dermis 116, a hypodermis, subcutaneous tissue, etc. A wound dressing 108 is disposed on the wound treatment site 102, e.g., the wound 103, and in some embodiments is operable to receive wound fluids from the wound treatment site 102 (e.g., gases and/or fluids emitted from the wound 103 and/or gases and/or fluids applied to the wound treatment site 102, the application of which may in some applications be controlled by the therapy unit 110.

In some embodiments, the wound dressing 108 may be substantially any relevant type of dressing to receive fluids from the patient. Further, in some applications the dressing may include a wound-interface dressing 118 or manifold (e.g., foam dressing, polyurethane porous foam, open-cell, reticulated foam, GranuFoam® material manufactured by Kinetic Concepts, Incorporated of San Antonio, Tex., GranuFoam Silver® material manufactured by Kinetic Concepts, Incorporated of San Antonio, Tex. V.A.C. WhiteFoam® dressing available from Kinetic Concepts, Inc. of San Antonio, Tex., hydrophilic foam, hydrophobic foams, bioresorbable materials, bandage, gauze, sponge, hydrogel, hydrocolloid, hydrofiber, occlusive dressing, scaffold, etc.) and a drape 120. The wound dressing 108 is configured to collect liquids and/or gases whether a wound is involved or not. In some embodiments, wound fluids, including liquids, from the wound treatment site 102 are delivered through a negative or reduced-pressure interface 122 to the site extraction tube 124.

The reduced-pressure interface 122 includes one or more wound fluid conduits 126 or reduced-pressure-supply conduit. In some implementations, the reduced-pressure interface 122 further includes one or more negative pressure sensor conduits 128 or pressure-assessment conduits. The wound fluid conduit 126 is fluidly coupled to the site extraction tube 124. Again, in some applications, the site extraction tube includes multiple lumens. Accordingly, in some embodiments the one or more wound fluid conduits 126 are fluidly coupled to one or more site wound fluid lumens 130 of the site extraction tube 124. Further, one or more negative pressure sensor conduits 128 are fluidly coupled to one or more negative pressure sensor lumen 132, which may be part of or separate from the site extraction tube 124. In one illustrative embodiment, the reduced-pressure interface 122 is a T.R.A.C.™ Pad or SensaT.R.A.C.™ Pad available from KCI of San Antonio, Tex. The reduced-pressure interface 122 may be any device capable of fluidly coupling the wound fluid lumen 130 to the wound dressing 108 to deliver reduced pressure to the wound treatment site and/or deliver and extract additional fluids to the wound treatment site. In some embodiments, the reduced-pressure interface 122 may further fluidly couple the one or more negative pressure sensor lumens 132 to a sealed space created by the drape 120.

In some embodiments, the in-line sampling assessment system 106 includes a housing 140, one or more input ports 133, one or more output ports 134, and one or more in-line sampling chambers 141-143. Typically, the input port 133 includes one or more wound fluid input ports 135, and the output port 134 includes one or more wound fluid output ports 136. The input port 133 and/or wound fluid input port 135 is secured with a housing 140 and fluidly coupled with an interior of the housing 140. Similarly, the output port 134 and/or the wound fluid output port 136 is secured with the housing 140 of the sampling assessment system 106 and is fluidly coupled with the interior of the housing and configured to fluidly couple with the wound fluid collection unit 170, typically through one or more unit extraction tubes 125. As such, a wound fluid path is established from the wound treatment site 102, through the sampling assessment system, and to the therapy unit 110 and/or wound fluid collection unit 170 for at least some of the wound fluid extracted from the wound treatment site.

In some implementations, one or more of the input ports 133 are configured to fluidly couple with the one or more site extraction tubes 124 to fluidly couple the site extraction tube with the in-line sampling assessment system 106. In some embodiments, the wound fluid input port 135 is configured to fluidly couple with at least the wound fluid lumen 130 extending from the wound treatment site 102. Some embodiments include one or more negative pressure sensor input ports 137 that are cooperated with the housing 140 and are configured to fluidly couple with one or more negative pressure sensor lumens 132, which may be part of or separate from the site extraction tube 124, and that extend from the wound treatment site 102. The one or more negative pressure sensor input ports 137 may be part of or separate from the input port 133. Similarly, some embodiments include one or more negative pressure sensor output ports 138 that are cooperated with the housing 140 and are configured to fluidly couple with one or more negative pressure sensor lumens 168, which may be part of or separate from the unit extraction tube 125, to fluidly couple with one or more external and separate negative pressure sensor systems 115, the therapy unit 110, and/or other pressure sensor systems. The one or more negative pressure sensor output ports 138 may be part of or separate from the output port 134. The negative pressure sensor system 115 can be implemented through substantially any relevant system capable of measuring or detecting a pressure. The negative pressure sensor system is configured to detect an actual reduced pressure at the tissue site, i.e., the applied pressure. In some embodiments, one or more of the wound fluid output ports 136 are configured to fluidly couple with one or more unit extraction tubes 125 establishing a fluid path with the reduced pressure source 112 and/or the wound fluid collection unit 170. Accordingly, the reduced pressure source 112 can establish a negative pressure at the wound treatment site 102 through the sampling assessment system 106.

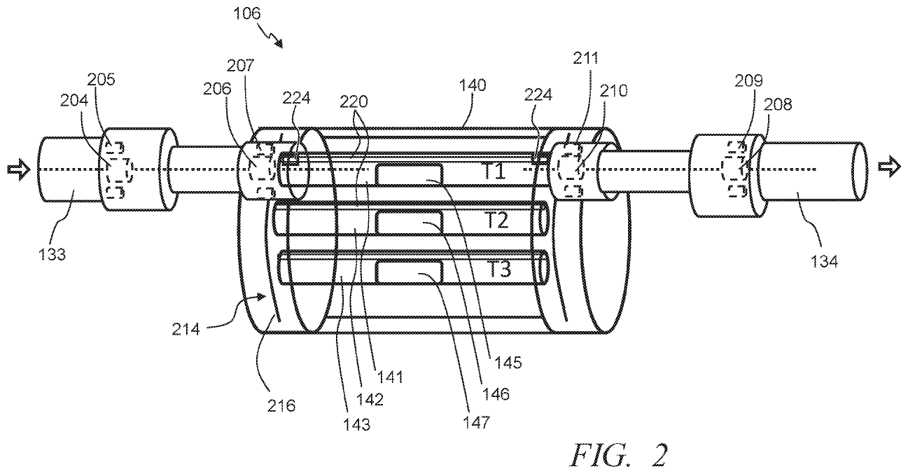

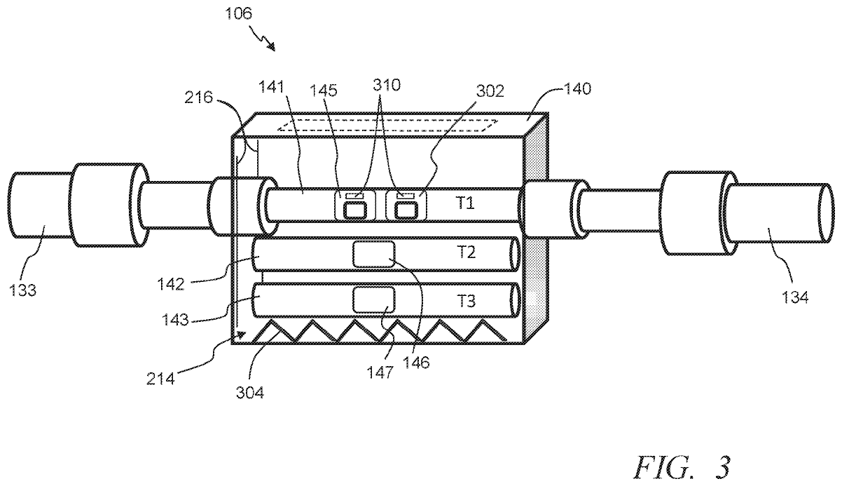

FIG. 2 illustrates a simplified block diagram, partially transparent view of an exemplary in-line sampling assessment system 106 with a rotational chamber switching system 214, in accordance with some embodiments. FIG. 3 illustrates a simplified block diagram, partially transparent view of an exemplary in-line sampling assessment system 106 with a linear chamber switching system 214, in accordance with some embodiments. Referring to FIGS. 1-3, the in-line sampling assessment system 106 typically includes at least one and often multiple in-line sampling chambers 141-143. The sampling chambers may include one or more theranostic sampling elements 145-147, 302. The in-line sampling chambers are positioned within the housing 140, which may include lateral sides and one or more walls extending between the sides to define an interior cavity or volume configured to receive the in-line sampling chamber 141-143.

The input and output ports 133, 134 are cooperated with the housing to provide a fluid path into and/or out of the volume of the housing 140. In some implementations, the sampling assessment system 106 may include one or more valves 204-211 and/or seals, one or more chamber switching systems 214 cooperated with the in-line sampling chambers 141-143. The in-line sampling chambers 141-143 can be configured to removably and fluidly couple between the input port 133 and the output port 134 in-line with the wound fluid path between the wound treatment site 102 and the therapy unit 110 and/or wound fluid collection unit 170 with the theranostic sampling element positioned so that at least some of the wound fluid contacts at least a portion of one or more theranostic sampling elements 145. The sampling chambers can be decoupled out of the wound fluid path while the sampling assessment system 106 maintains a negative pressure applied at the wound treatment site 102.

In some embodiments, one or more of the in-line sampling chambers 141-143 include one or more theranostic sampling elements 145-147. The sampling chambers 141-143 are configured to removably and fluidly couple between at least the wound fluid input port 135 of the input port 133 and the wound fluid output port 136 of the output port 134 to be in-line with a wound fluid path between the wound treatment site 102 and the wound fluid collection unit 170. When a sampling chamber 141 is fluidly coupled with the input port 133 and output port 134, a corresponding theranostic sampling element 145 is typically positioned so that at least some of the wound fluid that is feed into the sampling chamber 141 contacts at least a portion of the theranostic sampling element 145.

The sampling assessment system 106, in some applications, includes one or more fluid valves that fluidly and/or gaseously seal one or more portions of the sampling assessment system from other portions and/or external environments until opened. For example, the input port 133 may include one or more input valves 204-205 that are configured to be activated to open in response to the coupling with the site extraction tube 124 (e.g., one or more wound fluid input valves 204, one or more negative pressure sensor input valves 205, etc.). Additionally or alternatively, the input port 133 may include one or more switching valves 206-207 cooperated with an output end of the input port 133. Similarly, in some embodiments, the output port 134 may include one or more output valves 208-211. One or more of the output valves 208-209 may be positioned proximate to one or more exit conduits of the output port 134, and may be configured to be activated to open in response to the coupling with the unit extraction tube 125 (e.g., one or more wound fluid output valves 208, one or more negative pressure sensor output valves 209, etc.), triggered by an actuator, or otherwise activated to open or close. Additionally or alternatively, the output port 134 may include one or more switching valves 210-211 cooperated with an input end of the output port 134. In some implementations, one or more portions of the input ports 133 and output ports 134 may be extended and/or retracted, for example through rotation relative to threading, spring biasing, or the like, to simplify and/or enable coupling and decoupling with the site extraction tube 124, the unit extraction tube 125, one or more lumens of the site extraction tube 124 or unit extraction tube 125, the sampling chambers 141-143, other such coupling, or combination of such coupling.

The valves 204-211 can be implemented through one or more types of valves (e.g., ball-valve, solenoid valve, diaphragm valve, butterfly valve, etc.) that can be activated to open to establish a fluid flow, and/or to close to inhibit or prevent flow. For example, the input port 133 (and/or output port 134) may include spring biased valves that are spring bias closed and are opened through the insertion of a coupler of the site extraction tube 124 (or unit extraction tube 125) or one or more lumens of the site or unit extraction tubes 124, 125. Similarly, the one or more switching valves may be spring biased valves that are opened when a sampling chamber 141 is aligned with and fluidly coupled with an interior coupler of the input port 133 and/or output port 134. In some implementations, one or more of the valves may be controlled through rotation relative to threading. The opening may be triggered by pressure from the in-line sampling chamber, a motor triggered by the therapy unit 110, the activation of a locking mechanism of a chamber switching system and/or the in-line sampling assessment system 106, spring movement, or the like. In some implementations, one or more valves 204-211 may be implemented or replaced by one or more seals that may be punctured to establish a fluid and/or gas flow through the seal. For example, the switching valves 206-207, 210-211 may be replaced by one or more seals that are punctured by one or more needles of a sampling chamber 141-143, which may be spring loaded and released in response to an activation of a trigger, rotationally extended, or the like. Further, the seals may automatically reseal upon removal of the needle based on the nature of the seal as is known in the art. Additionally or alternatively, one or more of the input port, output port and the sampling chambers may include a luer taper, cannulae or the like, and corresponding one-way valves.

Further, in some embodiments, the in-line sampling chambers 141-143 may additionally or alternatively include one or more valves and/or seals. In some implementations, for example, a sampling chamber may include one or more input and output valves and/or seals, such as one or more wound fluid valves and one or more negative pressure sensor valves at each end of the sampling chamber. The valves may be biased to a closed state and opened in response to an alignment with the input and output ports 133, 134, a triggering mechanism, a rotation, or the like.

In some applications, the sampling chambers 141-143 include one or more negative pressure sensor conduits 220. The negative pressure sensor conduits 220 extend through the sampling chamber to align at opposite ends with the one or more negative pressure sensor input ports 137 and negative pressure output ports 138 of the input port 133 and output port 134 (and in some implementations align with one or more switching valves 207, 211), respectively. Further, the one or more negative pressure sensor conduits 220 in part establish one or more fluid paths between the negative pressure sensor input port 137 and the negative pressure sensor output port 138 along the in-line sampling chamber 141, and maintain one or more pressure sensor paths through the sampling assessment system 106 to couple with the therapy unit 110 and/or the negative pressure sensor system 115. The sampling chamber, in some applications, further includes pressure sensor couplers 224 that are configured to fluidly couple with the negative pressure sensor input and output ports 137, 138. Further, the one or more negative pressure sensor conduits 220 can be configured to extend between and fluidly couple one or more input pressure sensor couplers with one or more corresponding output pressure sensor couplers establishing one or more fluid pressure sensor paths between the negative pressure sensor input port 137 and the negative pressure sensor output port 138 along the in-line sampling chamber. In other embodiments, one or more bypass negative pressure sensor conduits are cooperated with the housing 140 of the sampling assessment system to fluidly couple with the negative pressure sensor input and output ports 137, 138 providing a negative pressure fluid path through the sampling assessment system 106. Alternatively or additionally, one or more sensor lumens are separate from and bypass the housing of the sampling assessment system 106.

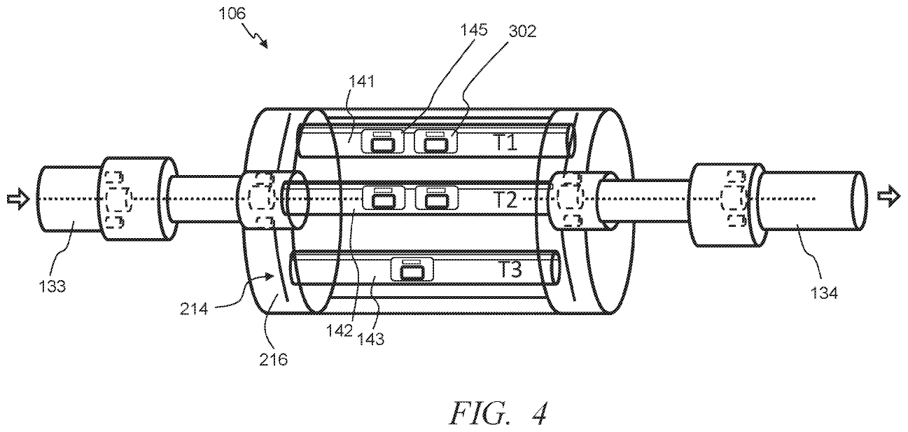

The sampling assessment system 106, in some embodiments, further includes one or more chamber switching systems 214 or mechanisms that enable the switching between the multiple sampling chambers 141-143. FIG. 4 illustrates a simplified block diagram, partially transparent view of an exemplary in-line sampling assessment system 106, in accordance with some embodiments, and following a rotation to align a subsequent sampling chamber 142 with the input and output ports 133, 134. Referring to FIGS. 1-4, in some embodiments, the chamber switching system includes one or more tracks 216 with which the sampling chambers are cooperated and that guide the sampling chambers 141-143 along a desired path relative to the input and output puts 133, 134. The chamber switching system 214 is configured to allow a first sampling chamber to decouple out of the wound fluid path while maintaining a negative pressure applied at the wound treatment site, and to allow a second sampling chamber to be moved into position to couple with the input and output ports and the wound fluid path. By switching between sampling chambers different samples of fluid can be collected, and/or different tests, diagnostics, etc. can be performed. Further, the switching allows for such collection and/or testing to be performed at different times (e.g., spaced over one or more threshold durations), performed at about the same time or within a relatively short period of time between switching with time between switching defined to enable fluid to effectively contact the theranostic sampling elements and/or collect sufficient sample quantities. Still further, the testing and/or diagnostics performed by the theranostic sampling elements 145-147 can be the same testing or diagnostic in one or more of the sampling chambers, or can provide different testing and/or diagnostics. Additional, the testing and/or diagnostics performed by different sampling chambers 141-143 can be the same testing or diagnostics, or can provide different testing and/or diagnostics.

In some embodiments, the chamber switching system 214 is secured with the housing 140. Multiple in-line sampling chambers 141-143 are movably cooperated with the chamber switching system 214. The chamber switching system enables the in-line sampling chambers to be moved relative to the input port 133 and the output port 134. The chamber switching system may rotationally move the sampling chambers, linearly move the sampling chambers, or otherwise move the sampling chambers to sequentially align with and provide at least a portion of the fluid flow path between the input and output ports. As illustrated in FIG. 3, some embodiments provide a linear track 216 within which each of the plurality of in-line sampling chambers 141-143 is movably cooperated and along which the plurality of in-line sampling chambers move to sequentially align and couple with the input port and the output port. In other embodiments, the chamber switching system 214 comprises a rotational track 216, which may be cooperated with the housing, and enables the plurality of in-line sampling chambers to be rotated relative to the housing in sequentially and fluidly coupling the plurality of sampling chambers with the input port and the output port.

Further, the chamber switching system, upon activation, is configured to cause an in-line sampling chamber to fluidly decouple from the input and output ports and cause another in-line sampling chamber to temporarily and fluidly couple with the input and output ports 133, 134 in-line with the wound fluid path while maintaining the negative pressure applied at the wound treatment site 102 and to enabling at least some wound fluid to contact at least a portion of the second theranostic sampling element.

Some embodiments utilize one or more valves and/or seals to maintain the pressure at the wound treatment site even as the sampling chambers are switched. Again, in some embodiments, one or more of the sampling chambers 141-143 may include one or more negative pressure sensor conduits 220, and/or one or more separate negative pressure sensor conduits may extend through the sampling assessment system 106 fluidly coupling the one or more negative sensor pressure input port 137 and the one or more negative pressure output ports 138. Accordingly, the valves may further open and close (or a seal punctured) relative to the switching between sampling chambers. In some implementations, the chamber switching system may include and/or activate valve opening and/or closing mechanisms to open or close one or more valves based on a switching between sampling chambers.

Some embodiments further include one or more locks, latches, biased pins and the like (generally referred to as locking system) that help to align the sampling chambers with the input and output ports 133, 134 during the switch between sampling chambers, and in some instances lock a sampling chamber in alignment with the input and output ports until unlocked and/or activated by the chamber switching system 214. The locking systems may be part of the chamber switching system 214 and activated by an external trigger that releases the chamber switching system 214 to allow the chamber switching system to move while moving the sampling chambers 141-143, and/or may unlock one or more sampling chambers to allow the sampling chambers to move relative to the chamber switching system 214, such as along the one or more tracks 216.

In some embodiments, the one or more tracks 216 are formed in and/or secured with the housing 140 and the sampling chambers are cooperated with the tracks. In other implementations, the sampling chambers may be cooperated with one or more carriages, chassis or other supports to maintain positioning of the sampling chambers 141-143 relative to the housing 140 and/or each other, and the carriage may be cooperated with the one or more tracks 216. For example, in some instances, a carriage may be generally a circular carriage that is rotationally cooperated with the housing through one or more axles, hubs, tracks, or the like. As the carriage rotates, the sampling chambers 141-143 are rotated. In other implementations the carriage may be configured to maintain the sampling chambers 141-143 in a linear alignment. One or more locking systems may engage and disengage from the carriage to inhibit and enable movement, respectively.

In some embodiments, the chamber switching system 214 includes one or more biasing elements (e.g., springs 304) that provide biasing on the one or more sampling chambers 141-143 and/or carriage, and that aids in switching between sampling chambers (e.g., in response to a release of a locking system, the spring exerts a force to move a first sampling chamber out of alignment with the input and output ports, and exerts a force to aid in moving and aligning a second sampling chamber). The carriage may include a frame that includes one or more gears and/or teeth to engage one or more gears and/or teeth that control movement of the carriage. Further, the carriage may include one or more slots, apertures, grooves, sockets, or the like into which a sampling chamber 141-143 is secured. In some applications, the slots may be configured to allow sampling chambers to be readily removed to be replaced by another sampling chamber (e.g., spring biasing may keep the sampling chamber in a desired position but allow an operator to move the sampling chamber against the biasing to free the sampling chamber from the slot) allowing reloading of the carriage with unexposed sampling chambers. The chamber switching system 214 and/or the sampling chambers 141-143 may include one or more spring biased alignment posts and/or other such alignment mechanisms that are configured to align with and/or engage one or more alignment recesses, apertures, bores, etc. In some implementations, the biasing may be overcome by rotational or linear force to move the sampling chambers, a trigger or button may overcome the biasing and/or move the alignment post out of an alignment aperture, a motor controlled by the therapy unit 110, etc.

In some embodiments, individual sampling chambers 141-143 may be fitted into individual slots within the chamber switching system, and/or a set of sampling chambers may be cooperated together (e.g., in a clip, carriage, frame or the like) that are inserted together within the housing 140 to cooperate with the chamber switching system 214. The chamber switching system may include one or more gears that engage teeth on the one or more tracks 216. An actuator may be cooperated external to the housing that allows an operator to initiate the switching between sampling chambers. For example, an operator may press or squeeze a release trigger that disengages a locking system and causes a rotation along the track of the sampling chambers (e.g., movement of a predefined amount consistent with alignment of a subsequent sampling chamber). in some embodiments, the chamber switching system comprises a rotational track 216 cooperated with the housing 140 enabling multiple in-line sampling chambers 141-143 to be rotated relative to the housing in decoupling and coupling the in-line sampling chambers with the input and output ports 133, 134 and the wound fluid path while maintaining the negative pressure.

In some applications, the release trigger may further cause a portion of the input and output ports 133, 134 and/or a portion of one or more sampling chambers 141-143 to retract to decouple a first sampling chamber from the flow path prior to switching, and upon subsequent alignment of a second sampling chamber to cause an extension of those one or more portions to fluidly couple and seal the second sampling chamber with the input and output ports. The release trigger, for example, may cause a rotation of an interior coupling portion, which is cooperated with the switching valves 206-207, 210-211, where the interior coupling portion that retracts and extents in response to the rotation enabling the decoupling and coupling with the sampling chambers as they are switched. In some embodiments, the sampling assessment system 106 may include one or more motors that are coupled with and controlled by the therapy unit 110 and activated to initiate the switching of the sampling chambers. In other implementations, the chamber switching system 214 is manually controlled by an operator, who may implement switching in response to instructions by the therapy unit 110, a doctor, a timing in accordance with a treatment protocol, an indication by one or more theranostic sampling elements 145-147, 302 of a condition of the wound fluid, or the like.

Still referring to FIGS. 1-4, as described above, the sampling chambers 141-143 may include one or more theranostic sampling elements 145-147, 302. The theranostic sampling elements are configured to provide feedback and information regarding the wound fluid, the state of a wound, a state of a treatment, and/or other such conditions. In some applications, each in-line sampling chamber 141-143 is configured to temporarily and releasably couple between the input port and the output port establishing at least a wound fluid path that is cooperated with one or more of the corresponding theranostic sampling elements 145-147, 302 of the in-line sampling chamber. Further, in some embodiments, when a sampling chamber 141 is fluidly coupled with the input port 133 and output port 134, one or more corresponding theranostic sampling elements 145 are typically positioned to expose the theranostic sampling element to the wound fluid extracted through the negative pressure from the wound treatment site 102.

In some embodiments, the wound fluid contacts at least some of the theranostic sampling element 145 and/or is transported by the theranostic sampling element or sampling chamber to contact one or more testing components of the theranostic sampling element. The contact of the fluid may include direct contact, extraction, collection, sampling, routing or the like. In some instances, one or more fluid contact systems 310 are utilized. For example, without limitation, the fluid contact system 310 of a theranostic sampling element 145 may include and/or employ one or more access windows or ports that allow fluid to enter and contact one or more testing systems, capillary action, wicking, absorption onto one or more chemicals and/or microspheres, submersion, collect sample of fluid (e.g., double chamber collection system), sequestration, chemical reaction, and/or other such mechanisms for capturing, sampling and/or testing the wound fluid. The chamber switching system is configured to enable each of the plurality of in-line sampling chambers 141-143 to be moved relative to the input port and the output port upon activation of the chamber switching system causing the plurality of in-line sampling chambers to sequentially couple with and provide the fluid path between the input port and the output port in sampling the wound fluid from the wound treatment site.

The testing components are configured to provide information about the condition of the wound fluid, the wound, the treatment, or the like. For example, a theranostic sampling element may include one or more pH testing components (e.g., one or more types of litmus paper) configured to provide an indication of a pH level of the wound fluid being extracted from the wound treatment site 102, protein detection component, bacterial detection component, glucose levels or concentrations, hemoglobin levels, other such testing, or combination of two or more of such testing. Numerous different conditions, sensing, testing, indications, diagnostics, theranostics can be implemented through one or more theranostic sampling elements of one or more sampling chambers (e.g., temperature, bacterial enzyme, cytokine-adsorbing columns (e.g., cytoSorb by Cytosorbents) cartridge with indicator, pH indicator, protease/matrix metalloproteinase (MMP), bacterial enzyme—biofilm, gene based detection, and other such testing).

Some theranostic sampling elements 145-147, 302 may be activated to initiate a chemical reaction and/or trigger a capture and/or transport of some of the wound fluid to initiate the diagnostic and/or theranostic processing. In some implementations, one or more of the individual theranostice sampling elements 145-147, 302 can provide processing while within the sampling assessment system 106 (e.g., an internal chemical reaction with one or more testing chemicals within the theranostic sampling element, sensors (e.g., temperature), dried blood spot, coatings on the inner walls of the sampling chamber and/or a portion of the theranostic sampling element, or the like). Other sampling chambers may be configured to stabilize, preserve and/or prepare wound fluid for subsequent analyses (e.g., through a subsequent activation, a subsequent introduction of one or more chemicals, removed from the sampling assessment system for subsequent processing, or the like.

Further, some theranostic sampling elements 145-147, 203 can be configured to provide visual indications, audible indication or the like of conditions of the treatment fluid, treatment process, wound site, or the like, while in-line with the fluid flow and/or while still in the sampling assessment system 106 yet transitioned out of the fluid flow. In some implementations, at least a portion of the casing of the sampling chamber is transparent and/or the casing includes one or more windows that allows a user to visually inspect at least a portion of the testing system to see the one or more visual indicators of the theranostic sampling element 147. Similarly, in some embodiments, at least a portion of the housing 140 of the sampling assessment system 106 is transparent and/or includes one or more windows that allows a user to see the one or more visual indicators of one or more sampling chambers. Further, in some instances the sampling results of multiple sampling chambers are simultaneously visible, which reflect conditions of the wound fluid corresponding to the sampling times of the theranostic sampling elements. For example, the housing 140 and casing of the sampling chambers may be formed of or include a transparent plastic material allowing a person to see, through the housing and casing, the visual indicator of the theranostic sampling element.

In some applications, theranostic sampling elements 145 include one or more visual diagnostic and/or theranostic result indicators that are configured to be triggered to visually indicate one or more conditions of the wound fluid. Two theranostic sampling elements may provide testing of the same condition, while in some instances different theranostic sampling elements provide information about different conditions. For example, a first theranostic sampling element may include a first theranostic visual result indicator that is configured to be triggered (e.g., exposed to the treatment fluid, an opening activated, the sampling chamber moved into position, etc.) and visually indicate a first condition of the wound fluid, while a second theranostic sampling element includes a second visual theranostic result indicator that is configured to be triggered to visually indicate a different second condition of the wound fluid. Additionally or alternatively, the theranostic sampling elements 145 may communicatively couple with the therapy unit 110, or other system to provide feedback to the unit. The therapy unit 110 can be configured to take one or more actions in response to the feedback (e.g., generate one or more visual or audible alerts, communicate a notification or alert (e.g., via email, text message, instant message, cellular, local area network communication, dedicated communication, etc.), record status information, modify a treatment protocol (e.g., cause an increase in pressure, cause a reduction in pressure, cause a fluid to be applied to the wound, cause a reduction of or stopping a fluid being applied, etc.), and/or other such actions).

As described above, a single sampling chamber 141 may include multiple theranostic sampling elements 145, 302. The multiple theranostic sampling elements may be positioned so that wound fluid contacts at least a portion of one or more of the theranostic sampling elements at the same time. Similarly, a single theranostic sampling element may include multiple visual theranostic result indicators that can be exposed to the wound fluid and that may indicate at the same time different conditions of the wound fluid (e.g., presence or absence of a bacterial enzyme, and pH level). Further, one or more of the theranostic sampling elements 145, 302 may provide different indicators, such as visual indicators visually indicating different conditions of the wound fluid at the same time (e.g., first and second different conditions visually indicated by separate first and second visual indicators). In other embodiments, the multiple theranostic sampling elements within a single sampling chamber 141 may be exposed to the treatment fluid at different times.

Additionally, some theranostic sampling elements 145-147, 302 provide feedback within a relatively short period of time (e.g., within seconds, or tens of minutes) providing substantially real-time feedback of the wound fluid, wound site, and the like. Still further, some theranostic sampling elements continue to provide feedback over time indicating changes over time of the conditions based on the wound fluid. Having the option of sampling chambers being processed at near real-time allows for flexibility in terms of batch processing, rapid adjustment in treatment protocols, allows for families of target markers to be clustered in specific sampling chambers, and the like, while a treatment protocol can continue to be implemented and/or adjusted. Further, in some embodiments the feedback provided by the theranostic sampling elements without interrupting a negative pressure at the wound treatment site 102. Additionally, the sampling assessment system 106 can be implemented in-line with the therapy unit 110 providing feedback regarding the wound site, the state of the wound and/or the progress of wound treatment while the wound treatment protocol is in progress and without having to remove the wound dressing 108, drape 120 and/or reduced-pressure interface 122.

In some embodiments, one or more of the sampling chambers 141-143, 203 or theranostic sampling elements are connected to an external testing device for sample processing preparation and/or evaluation (e.g., a single port per device or per sampling chamber). This allows samples to be evaluated using ancillary systems (e.g., lateral flow, real-time detection polymerase chain reaction assay (RTD-PCR), etc.). Similarly, some of the sampling assessment systems and/or sampling chambers are temperature controlled to reduce risk of decomposition of one or more sample acquired through the sampling chamber and/or for processing of samples. As described above, in some embodiments, the sampling assessment system includes the one or more switching valves 206-207, 210-211 in the input and output ports 133, 134 and/or in the sampling chambers 141-143 to maintain the desired treatment pressure at the wound treatment site 102 while allowing switching between two or more sampling chambers in-line with the NPWT line (e.g., the extraction tubes 124, 125), which typically would otherwise have to include a temporary stopping of NPWT therapy.

Further, the housing 140 of the sampling assessment system 106, in some implementations, includes one or more doors 312, openings, hatches, or the like that allows one or more in-line sampling chambers 141-143 to be removable from the housing 140 and/or replaced with another sampling chamber. In other implementations, one or more of the sampling chambers 141-143 are secured within the housing 140, while one or more of the theranostic sampling elements 145-147, a portion of a theranostic sampling element, or a sub-chamber may be configured to be removed, and/or other theranostic sampling elements, portion of a theranostic sampling element, or sub-chamber to be inserted. Further, in some applications, a sampling chamber and/or theranostic sampling element (or portion thereof) can be removed while the negative pressure applied at the wound treatment site 102 is maintained after the sampling chamber and/or the theranostic sampling element has been decoupled from the wound fluid path. Additionally or alternatively, some embodiments enable the removal of a sampling chamber and/or a theranostic sampling element (or portion thereof) while ensuring that therapy unit 110 and/or negative pressure sensor system 115 continue to sense the pressure at the wound treatment site 102 and wound dressing 108 to enable continued pressure regulation through a control circuit of the therapy unit 110 and/or reduced pressure source 112. In some applications, the pressure at the wound site is maintained through one or more 220 negative pressure sensor conduits 220 of a sampling chamber fluidly coupled with the input and output ports, one or more separate sensor conduits of the housing 140, one or more external bypass sensor lumens bypassing the sampling assessment system 106, or the like.

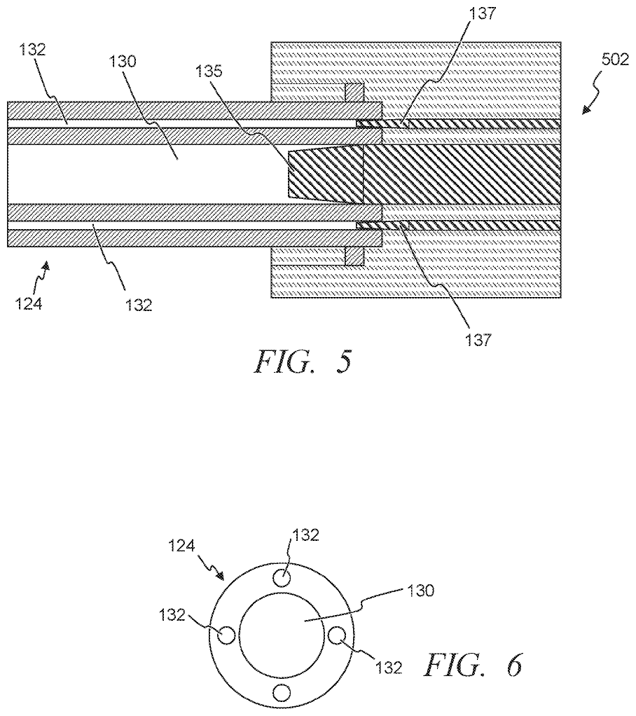

FIG. 5 illustrates a simplified cross-sectional view of a portion of an exemplary input port 133, in accordance with some embodiments. FIG. 6 illustrates a simplified plane view of an exemplary site extraction tube 124 and/or a unit extraction tube 125, in accordance with some embodiments. Referring to FIGS. 1-6, as described above, in some implementations the site extraction tube 124 and/or the unit extraction tube 125 may be a multi-lumen tube. In some applications, one or more lumens may be wound fluid lumens 130 through which negative pressure is applied at the wound treatment site 102 and through which one or more fluids are extracted from the wound treatment site. In some instances, the wound fluid lumen 130 may further be utilized to apply one or more fluids to the wound treatment site 102, such as fluid applied by the therapy unit 110 or other system fluidly coupled with the wound treatment site. Further, one or more negative pressure sensor lumens 132 may be cooperated with the wound fluid lumen 130, and in some instances are formed with the wound fluid lumen as a single tube. For example, multiple negative pressure sensor lumens 132 may be formed about a centrally located wound fluid lumen, such as the multi-lumen tubing of the SensaT.R.A.C.™ technology from KCI of San Antonio, Tex.

The input port 133, in some embodiments, is a multi-lumen input port and comprises an input fluid splitter 502 comprising, at an input end, one or more wound fluid input ports 135 and at least one negative pressure sensor input port 137. The wound fluid input port 135 illustrated in FIG. 5 is configured to fluidly couple and seal with a wound fluid lumen 130 of an extraction tube (e.g., site extraction tube 124) that includes both the wound fluid lumen 130 and at least one negative pressure sensor lumen 132. Further, in some applications, each of the negative pressure sensor input ports 137 is configured to fluidly couple and seal with at least one of the one or more negative pressure sensor lumens 132 of the site extraction tube 124 extending from the wound treatment site.

Similarly, in some embodiments, the output port 134 is implemented through a multi-lumen fluid splitter similar to the input fluid splitter 502, and comprises at least one wound fluid output port 136 that, at an output end, is configured to fluidly couple and seal with a wound fluid lumen 164 of a unit extraction tube 125 that extends to a therapy control unit 110 or other system. Further, the output port 134 can include one or more negative pressure sensor output ports 138 each configured to fluidly couple and seal with at least one of the one or more negative pressure sensor lumens 168 of the unit extraction tube 125.

Some embodiments provide the in-line NPWT sampling assessment system that includes at least one sampling assessment system 106 as a multi-chambered fluid collection device that is inserted in-line with the NPWT circuit (e.g., dressing, connector, and tubing coupled with the housing 140) without compromising the integrity of the NPWT system (e.g., SensaT.R.A.C. system) and/or instillation system, which may be accomplished by, but not restricted to, having a line splitter, resulting in one or more tubing or housings for maintaining continuity of one or more lumens, including pressure sensor lumens 132. The sampling assessment system allows the multiple sampling chambers 141-143 to be switched without interrupting the NPWT for the set-up, fluid collection and/or fluid sampling. Further, the sampling chambers 141-143 cooperated with housing 140 may or may not be within an inner cartridge. As described above, in some applications each individual sampling chamber or inner theranostic sampling elements and/or diagnostic cartridges containing individual chambers and sub-chambers can be removed and/or replaced without disrupting therapy and without removing the wound dressing 108.

In some implementations, the sampling chambers 141-143 and/or theranostic sampling elements 145-147, 203 are configured to be in series or in parallel within a the sampling assessment system 106. When in parallel, the fluid path is split to direct some fluid into one or more of the sampling chambers, and in some instances simultaneously directing fluid into two or more sampling chambers. One or more sampling chambers 141-143 may include one or more theranostic sampling elements 145-147, 203 or sub-chambers within the fluid flow path that allow for fluid processing or multiplexing. As described above, in some embodiments, one or more sampling chambers 141-143 can be repositioned within the sampling assessment system 106 such that wound exudate, gases and/or one or more other wound fluids can be collected and/or sampled multiple times or at different time points.

In some embodiments, one or more of the theranostic sampling elements 145-147, 203 are configured to provide in-situ processed of the fluid. One or more theranostic sampling elements may stabilize, preserve and/or prepare the fluid for subsequent analyses. Additionally or alternatively, one or more theranostic sampling elements may sense and/or test the fluids (e.g., with sensors/chemicals placed within the theranostic sampling element (e.g., litmus paper for pH, dried blood spot,) with coatings on inner walls of the sampling chamber and/or the theranostic sampling element, etc.), may collect the fluid to be removed and then further processed, archived or discarded, and/or other such processing.

The sampling chambers and theranostic sampling elements can provide for fluid processing at a time while the treatment is being implemented allows for flexibility in terms of batch processing if needed, and allows for families of target markers to be clustered in specific cartridges. In some embodiments, one or more of the sampling chambers 141-143 and/or theranostic sampling elements 145-147, 203 may connect to an external device for sample processing preparation and/or evaluation (e.g., via a single port per device or per chamber or theranostic sampling element). This allows samples to be evaluated using ancillary systems (e.g., lateral flow, real-time detection polymerase chain reaction (RTD-PCR, etc.). Further, in some embodiments, one or more of the sampling chambers 141-143 and/or theranostic sampling elements 145-147, 203 are temperature controlled, which may reduce a risk of decomposition of the sample and/or provide for at least part of the processing of samples. As described above, in some embodiments, the sampling assessment system 106 enables sampling chambers 141-143 to be switched. Some embodiments include switching valves 206-207, 210-211 that allow for insertion and removal of the sampling chambers and/or theranostic sampling elements into the NPWT line, which would otherwise typically include the clamping of the line, which would then trigger alarms and/or include a temporary stopping of therapy.

FIG. 7 illustrates a simplified flow diagram of an exemplary process 700 of implementing a wound treatment, such as a negative pressure wound therapy, in accordance with some embodiments. In step 702, a wound fluid, transported by a wound fluid lumen 130 from the wound treatment site 102, is enabled to be directed to a first in-line sampling chamber 141 that is removably and fluidly coupled with the wound fluid lumen. In some instances, a theranostic sampling element 145 within the in-line first sampling chamber is exposed to the wound fluid. Further, in some embodiments, the wound fluid is transported by the wound fluid lumen 130 in response to a negative wound therapy pressure applied through the wound fluid lumen at the wound treatment site. In some embodiments, the sampling assessment system includes one or more input ports 133 and output ports 134 that provide a coupling with external lumens, and further provide fluid communication two the one or more sampling chambers. The input and output ports, in some implementations, includes one or more valves to enable coupling with the sampling chambers and direct the fluid to the coupled sampling chamber.

In step 704, a switching from the first in-line sampling chamber 141 to a second in-line sampling chamber 142 is enabled. In same embodiments, a user can pull out the first sampling chamber from the housing 140 and replace the first sampling chamber with the second sampling chamber, while other implementations a chamber switching system 214 is utilized. The first and second sampling chambers are cooperated with the chamber switching system, and the chamber switching system can switch from the first in-line sampling chamber to the second in-line sampling chamber decoupling the first in-line sampling chamber from a fluid path of the wound fluid and removably and fluidly coupling the second in-line sampling chamber with the first wound fluid lumen 130.

In step 706, the wound fluid is enabled to be directed to the second in-line sampling chamber to expose a second theranostic sampling element 146 within the second in-line sampling chamber to the wound fluid. As described above, in some embodiments, the theranostic sampling element includes one or more fluid contact systems 310 that enable contact and/or capture of fluid (e.g., access windows or ports, capillary action, wicking, absorption, submersion, and/or such mechanisms to enable capturing, sampling and/or testing the wound fluid). Some embodiments enable the exposure of the wound fluid to the first theranostic sampling element 145 to cause a triggering of a visual result indicator to visually indicate a condition of the wound fluid.

In some embodiments, a pressure sensor path is substantially maintained between the wound treatment site 102 and the therapy unit 110 and/or a pressure sensor system 115 through a multi-lumen input port 133 (e.g., with potentially only minor variations due to differences in pressure between the interior of the sampling chambers, minor leaking, etc.). In some embodiments, one or more valves (e.g., switching valves 206-207, 210-211, pressure sensor couplers valves, input valves 204-205, output valves 208-209, etc.) can be activated to at least maintain the pressure for at least a relatively short period of time while a first sampling chamber 141 is switch out and a second sampling chamber 142 is switched in to continue to provide at least the fluid path through the sampling assessment system 106. In some embodiments, the switching between sampling chambers is relatively quick (typically less than five seconds, and often less than one second). Accordingly, the pressure is substantially maintained during that relatively short switching duration. Additionally or alternatively, the chamber switching system 214 may include one or more surfaces that abut with and/or otherwise seal the output of the input port 133 and/or the input of the output port 134 while switching between sampling chambers, and the sealing substantially maintains the pressure at least during the switching. The input port can fluidly couple an input end of a pressure sensor input port 137 of the input port 133 with a pressure sensor lumen 132 extending from the wound treatment site 102. Similarly, the output port can include a pressure sensor output port 138 as part of a multi-lumen output port. An output end of the pressure sensor output port 138 can couple with a pressure sensor lumen 168 extending to the pressure sensor system 115. Further, some embodiments maintain the pressure sensor path and enable the pressure sensor path to fluidly extend through the first in-line sampling chamber 141 through a releasable and fluid coupling between an output end of the pressure sensor input port 137 of the multi-lumen input port and a first pressure sensor coupler 224 of the first in-line sampling chamber, along a negative pressure sensor conduit 220 of the first in-line sampling chamber extending between and fluidly coupling the first pressure sensor coupler with a second pressure sensor coupler 224.

Some embodiments implement the fluid sampling and/or testing as part of a treatment protocol that includes multiple extended negative pressure cycles (e.g., 1 hour, 2 hours, 4 hours, 10 hours, etc.). One or more sampling phase can be implemented during and/or between negative pressure cycles. In some implementations, the process can detect a termination of an extended negative pressure cycle of a negative pressure wound therapy protocol, and initiate a sampling phase of the negative pressure wound therapy protocol following the termination of the extended negative pressure cycle. In some instances, the wound fluid can be directed to the first sampling chamber 141 over a duration that is typically less than a duration of the extended negative pressure cycle. A subsequent extended negative pressure cycle and/or an instillation of topical wound solutions (e.g., antimicrobial, antiseptic, etc.) of the negative pressure wound treatment protocol can subsequently be initiated.

The flow of wound fluid may be controlled in some embodiments through one or more valves. Some embodiments control a valve 252, cooperated with the first wound fluid lumen, to expose the first wound fluid lumen to an air source and inducing an increased rate of assent of at least some wound fluid along the first wound fluid lumen and into the first in-line sampling chamber 141. In some implementations, the therapy unit 110 is electrically and/or communicatively coupled with an activation system of the valve 252 that controls the opening and closing of the valve, and allowing the therapy unit to control the valve and the sampling phase.

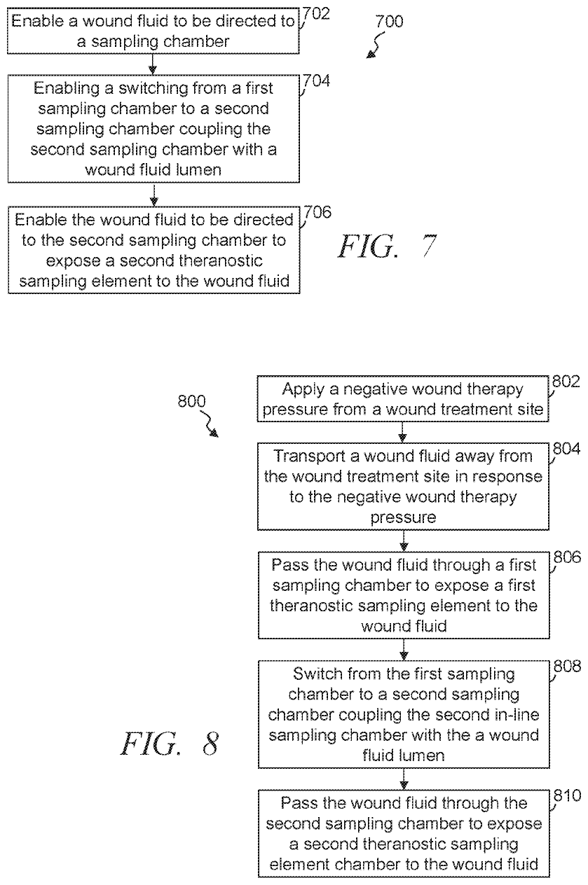

FIG. 8 illustrates a simplified flow diagram of an exemplary process 800 of implementing a treatment therapy, such as a negative pressure wound therapy, in accordance with some embodiments. In step 802, a negative wound therapy pressure is applied through a first wound fluid lumen 130 extending from a wound treatment site 102. In step 804, at least some of the wound fluid is transported away from the wound treatment site via the first wound fluid lumen in response to the negative wound therapy pressure. In step 806, the wound fluid is passed through a first in-line sampling chamber 141 that is removably and fluidly coupled with the first wound fluid lumen 130 to expose a first theranostic sampling element 145 within the first in-line sampling chamber to the wound fluid.

In step 808, a chamber switching system 214 coupled with the first in-line sampling chamber 141 and a second in-line sampling chamber 142 switches from the first in-line sampling chamber 141 to the second in-line sampling chamber 142 to decouple the first in-line sampling chamber from a fluid path of the wound fluid and removably and fluidly couples the second in-line sampling chamber with the first wound fluid lumen. In step 810, the wound fluid is passed through the second in-line sampling chamber to expose a second theranostic sampling element 146 within the second in-line sampling chamber to the wound fluid. In some embodiments, the therapy unit 110 electrically and/or communicatively couples with the chamber switching system 214 and activates the switching in accordance with the treatment protocol and/or the sampling phase. For example, the chamber switching system may include one or more motors, magnetic systems, biasing, or the like that cause the activation and/or movement of at least part of the chamber switching system. In other implementations, the therapy unit may generate a notification, alert, alarm or the like (e.g., through a user interface, audible alert, text message, cellular call, etc.) that notifies a user to manually activate the sampling assessment system 106 and/or the chamber switching system 214 to implement the transition between the sampling chambers.

As described above, in some embodiments, the theranostic sampling elements may provide feedback regarding the wound fluid extracted from the wound treatment site 102, the state of the treatment and/or whether modifications to a treatment protocol are to be implemented. In some embodiments, a user can enter results from a theranostic sampling element 145-147 into the therapy unit 110 and/or other computing device (e.g., computer, smartphone, tablet, etc.) that can communicate with the therapy unit 110 to provide information to and/or implement adjustments to the therapy unit (e.g., adjustments that cause adjustments to the wound treatment protocol). Some theranostic sampling elements 145-147 may be communicatively coupled with the therapy unit 110 and/or other computing device through wired (e.g., through a direct coupling or through a network (e.g., local area network (LAN), wide area network (WAN), Internet, etc.) and/or wireless coupling (e.g., Bluetooth, Wi-Fi, cellular, etc.).

Based on the information provided from the theranostic sampling elements 145-147 and/or other testing systems that test fluid captured by a sampling chamber 141-143, the treatment protocol may be confirmed and/or adjusted. Again, in some instances, the theranostic sampling elements can provide data to the therapy unit 110, while in other instances a user may enter data into the therapy unit. In still other instances, a user may select one or more options on the therapy unit based on the indications and/or feedback received from the one or more theranostic sampling elements. Options may be pre-programmed and displayed on a user display of the therapy unit corresponding to different expected results from one or more of the theranostic sampling elements 145-147.

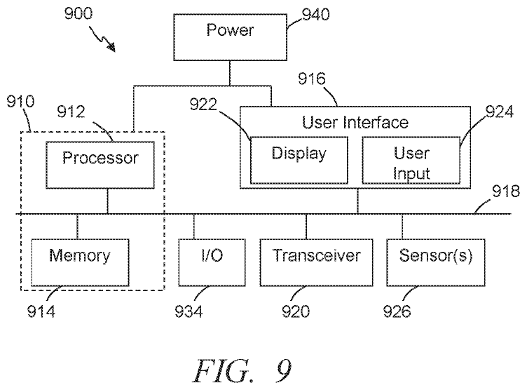

The control and/or processing methods, processes, techniques, systems, devices, services, servers, sources and the like described herein may be utilized, implemented and/or run on many different types of devices and/or systems. Referring to FIG. 9, there is illustrated a system 900 that may be used for any such implementations, in accordance with some embodiments. One or more components of the system 900 may be used for implementing any unit, system, circuit, apparatus or device mentioned above or below, or parts of such units, systems, circuits, apparatuses or devices, such as for example any of the above or below mentioned therapy units 110, reduced pressure source 112, wound fluid collection unit 170, negative pressure sensor system 115, user device (e.g., smartphone, tablet, computer, etc.), sensor systems, control circuits, and the like. However, the use of the system 900 or any portion thereof is certainly not required.

By way of example, the system 900 may comprise a controller or processor module 912, memory 914, a user interface 916, and one or more communication links, paths, buses or the like 918. A power source or supply 940 is included or coupled with the system 900. The controller 912 can be implemented through one or more processors, microprocessors, central processing unit, logic, local digital storage, firmware and/or other control hardware and/or software, and may be used to execute or assist in executing the steps of the processes, methods and techniques described herein, and control various communications, programs, content, listings, services, interfaces, etc. Further, in some embodiments, the controller 912 can be part of a control system 910 and/or implemented through one or more processors with access to one or more memory 914. The user interface 916 can allow a user to interact with the system 900 and receive information through the system. In some instances, the user interface 916 includes a display 922 and/or one or more user inputs 924, such as a remote control, keyboard, mouse, track ball, game controller, buttons, touch screen, etc., which can be part of or wired or wirelessly coupled with the system 900.

In some embodiments, the system 900 further includes one or more communication interfaces, ports, transceivers 920 and the like allowing the system 900 to communication over a communication bus, a distributed network, a local network, wide area network, the Internet, communication link 918, other networks or communication channels with other devices and/or other such communications or combinations thereof. Further the transceiver 920 can be configured for wired, wireless, optical, fiber optical cable or other such communication configurations or combinations of such communications.

The system 900 comprises an example of a control and/or processor-based system with the controller 912. Again, the controller 912 can be implemented through one or more processors, controllers, central processing units, logic, software and the like. Further, in some implementations the controller 912 may provide multiprocessor functionality.

The memory 914, which can be accessed by the controller 912, typically includes one or more processor readable and/or computer readable media accessed by at least the controller 912, and can include volatile and/or nonvolatile media, such as RAM, ROM, EEPROM, flash memory and/or other memory technology. Further, the memory 914 is shown as internal to the system 910; however, the memory 914 can be internal, external or a combination of internal and external memory. Similarly, some or all of the memory 914 can be internal, external or a combination of internal and external memory of the controller 912. The external memory can be substantially any relevant memory such as, but not limited to, one or more of flash memory secure digital (SD) card, universal serial bus (USB) stick or drive, other memory cards, hard drive and other such memory or combinations of such memory. The memory 914 can store code, software, executables, scripts, data, content, programming, programs, textual content, identifiers, log or history data, user information, thresholds, instructions, and the like.

One or more of the embodiments, methods, processes, approaches, and/or techniques described above or below may be implemented in one or more computer programs executable by a processor-based system. By way of example, such a processor based system may comprise the processor based system 900, a computer, tablet, smartphone, etc. Such a computer program may be used for executing various steps and/or features of the above or below described methods, processes and/or techniques. That is, the computer program may be adapted to cause or configure a processor-based system to execute and achieve the functions described above or below. For example, such computer programs may be used for implementing any embodiment of the above or below described steps, processes or techniques to provide wound therapy treatment, evaluate a wound, and/or evaluate a wound treatment protocol. As another example, such computer programs may be used for implementing any type of tool or similar utility that uses any one or more of the above or below described embodiments, methods, processes, approaches, and/or techniques. In some embodiments, program code modules, loops, subroutines, etc., within the computer program may be used for executing various steps and/or features of the above or below described methods, processes and/or techniques. In some embodiments, the computer program may be stored or embodied on a computer readable storage or recording medium or media, such as any of the computer readable storage or recording medium or media described herein.

Accordingly, some embodiments provide a processor or computer program product comprising a medium configured to embody a computer program for input to a processor or computer and a computer program embodied in the medium configured to cause the processor or computer to perform or execute steps comprising any one or more of the steps involved in any one or more of the embodiments, methods, processes, approaches, and/or techniques described herein.

Some embodiments provide apparatuses, systems and corresponding methods to provide in-line therapy theranostic sampling and/or diagnostics. Further, some embodiments provide in-line negative pressure wound therapy (NPWT) sampling assessment systems, comprising: a housing; a wound fluid input port secured exterior to the housing and fluidly coupled with an interior of the housing, wherein the wound fluid input port is configured to fluidly couple with a first wound fluid lumen extending from a wound treatment site; a wound fluid output port secured exterior to the housing and fluidly coupled with the interior of the housing and configured to fluidly couple with a NPWT control unit; a first in-line sampling chamber positioned within the housing and comprising a first theranostic sampling element, wherein the first in-line sampling chamber is configured to: removably and fluidly couple between the input port and the output port in-line with a wound fluid path between the wound treatment site and the NPWT control unit with the first theranostic sampling element positioned so that at least some of the wound fluid contacts at least a portion of the first theranostic sampling element; and decouple out of the wound fluid path without interrupting a negative pressure treatment process applied at the wound treatment site.

In some embodiments, in-line NPWT sampling assessment systems comprise: an input port; an output port; a chamber switching system; and a plurality of in-line sampling chambers each cooperated with the chamber switching system and wherein each of the plurality of in-line sampling chambers comprises: a theranostic sampling element; and a fluid through path, wherein each in-line sampling chamber is configured to: temporarily and releasably couple between the input port and the output port establishing at least a wound fluid path that is cooperated with the theranostic sampling element; and expose the theranostic sampling element to a wound fluid extracted through negative pressure from a wound treatment site; and wherein the chamber switching system is configured to enable each of the plurality of in-line sampling systems to be moved relative to the input port and the output port upon activation causing the plurality of in-line sampling systems to sequentially couple with and provide the fluid path between the input port and the output port in sampling the wound fluid from the wound treatment site.

Additionally, some embodiments provide methods of implementing a negative pressure wound therapy, comprising: enabling a wound fluid, transported by a first wound fluid lumen in response to a negative wound therapy pressure applied through the first wound fluid lumen from a wound treatment site, to be directed to a first in-line sampling chamber that is removably and fluidly coupled with the first wound fluid lumen to expose a first theranostic sampling element within the first in-line sampling chamber to the wound fluid; enabling a switching, through a chamber switching system with which the first in-line sampling chamber and an in-line second sampling chamber are cooperated, from the first in-line sampling chamber to the second in-line sampling chamber decoupling the first in-line sampling chamber from a fluid path of the wound fluid and removably and fluidly coupling the second in-line sampling chamber with the first wound fluid lumen; and enabling the wound fluid to be directed to the second in-line sampling chamber to expose a second theranostic sampling element within the second in-line sampling chamber to the wound fluid.

Some embodiments provide methods of implementing a negative pressure wound therapy, comprising: applying a negative wound therapy pressure through a first wound fluid lumen from a wound treatment site; transporting a wound fluid away from the wound treatment site via the first wound fluid lumen in response to the negative wound therapy pressure; passing the wound fluid through a first in-line sampling chamber that is removably and fluidly coupled with the first wound fluid lumen to expose a first theranostic sampling element within the first in-line sampling chamber to the wound fluid; switching, using a chamber switching system coupled with the first in-line sampling chamber and a second in-line sampling chamber, from the first in-line sampling chamber to the second in-line sampling chamber to decouple the first in-line sampling chamber from a fluid path of the wound fluid and removably and fluidly coupling the second in-line sampling chamber with the first wound fluid lumen; and passing the wound fluid through the second in-line sampling chamber to expose a second theranostic sampling element within the second in-line sampling chamber to the wound fluid.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

What is claimed is:

1. An in-line negative pressure wound therapy (NPWT) sampling assessment system, comprising:
a housing;
a wound fluid input port secured exterior to the housing and fluidly coupled with an interior of the housing, wherein the wound fluid input port is configured to fluidly couple with a first wound fluid lumen extending from a wound treatment site;
a wound fluid output port secured exterior to the housing and fluidly coupled with the interior of the housing and configured to fluidly couple with a NPWT control unit;
a first in-line sampling chamber positioned within the housing and comprising a first theranostic sampling element, wherein the first in-line sampling chamber is configured to:
removably and fluidly couple between the input port and the output port in-line with a wound fluid path between the wound treatment site and the NPWT control unit with the first theranostic sampling element positioned so that at least some of the wound fluid contacts at least a portion of the first theranostic sampling element; and
decouple out of the wound fluid path without interrupting a negative pressure treatment process applied at the wound treatment site.

2. The system of claim 1, further comprising:
a chamber switching system secured with the housing; and
a second in-line sampling chamber, wherein the first in-line sampling chamber and the second in-line sampling chamber are movably cooperated with the chamber switching system, wherein the second in-line sampling chamber comprises a second theranostic sampling element;
wherein the chamber switching system is configured to enable the first and second in-line sampling systems to be moved relative to the input port and the output port and upon activation to cause the first in-line sampling chamber to fluidly decouple from the input and output ports and cause the second in-line sampling chamber to temporarily and fluidly couple with the input and output ports in-line with the wound fluid path while maintaining the negative pressure applied at the wound treatment site enabling at least some wound fluid to contact at least a portion of the second theranostic sampling element.

3. The system of claim 2, wherein the first theranostic sampling element further comprises a first visual theranostic result indicator that is configured to be triggered to visually indicate a first condition of the wound fluid; and
the second theranostic sampling element further comprises a second visual theranostic result indicator that is configured to be triggered to visually indicate a different second condition of the wound fluid.

4. The system of claim 3, wherein first in-line sampling chamber comprises the first theranostic sampling element and a third theranostic sampling element positioned so that the wound fluid contacts at least a portion of the third theranostic sampling element while the wound fluid contacts at least the portion of the first theranostic sampling element, wherein the third theranostic sampling element comprises a third visual theranostic result indicator configured to be triggered to visually indicate a third condition of the wound fluid at the same time the first visual indicator is visually indicating the first condition of the wound fluid.

5. The system of claim 2, wherein the chamber switching system comprises a rotational track cooperated with the housing enabling the first and second in-line sampling chambers to be rotated relative to the housing in decoupling and coupling the first and second in-line sampling chambers with the input and output ports and the wound fluid path while maintaining the negative pressure.

6. The system of claim 2, wherein the first in-line sampling chamber is removable from the housing while maintaining the negative pressure applied at the wound treatment site and after having been decoupled from the wound fluid path.

7. The system of claim 1, wherein the first theranostic sampling element further comprises a visual theranostic result indicator that is configured to be triggered to visually indicate a condition of the wound fluid.

8. The system of claim 1, further comprising:
a negative pressure sensor input port cooperated with the housing and configured to fluidly couple with at least one negative pressure sensor lumen extending from the wound treatment site; and
a negative pressure sensor output port cooperated with the housing and configured to fluidly couple with an external and separate negative pressure sensor system.

9. The system of claim 8, wherein the first in-line sampling chamber comprises a first pressure sensor coupler, a second pressure sensor coupler, and a first negative pressure sensor conduit extending between and fluidly coupling the first pressure sensor coupler with the second pressure sensor coupler establishing a fluid path between the negative pressure sensor input port and the negative pressure sensor output port along the first in-line sampling chamber.

10. The system of claim 1, wherein the input port comprises an input fluid splitter comprising an input wound fluid conduit and at least one input negative pressure sensor conduit, wherein the input wound fluid conduit is configured to fluidly couple and seal with the first wound fluid lumen of a first extraction tube comprising the first wound fluid lumen and at least one negative pressure sensor lumen;
wherein the at least one negative pressure sensor conduit is configured to fluidly couple and seal with the at least one negative pressure sensor lumen of the first extraction tube; and
wherein the output port comprises an output wound fluid conduit configured to fluidly couple and seal with a second wound fluid lumen of a second extraction tube, and at least one output negative pressure sensor conduit configured to fluidly couple and seal with at least one negative pressure sensor lumen of the second extraction tube.

11. An in-line negative pressure wound therapy (NPWT) sampling assessment system, comprising:
an input port;
an output port;
a chamber switching system; and
a plurality of in-line sampling chambers each cooperated with the chamber switching system and wherein each of the plurality of in-line sampling chambers comprises:
a theranostic sampling element; and
a fluid through path,
wherein each in-line sampling chamber is configured to:
temporarily and releasably couple between the input port and the output port establishing at least a wound fluid path that is cooperated with the theranostic sampling element; and
expose the theranostic sampling element to a wound fluid extracted through negative pressure from a wound treatment site; and
wherein the chamber switching system is configured to enable each of the plurality of in-line sampling systems to be moved relative to the input port and the output port upon activation causing the plurality of in-line sampling systems to sequentially couple with and provide the fluid path between the input port and the output port in sampling the wound fluid from the wound treatment site.

12. The system of claim 11, wherein:
the input port comprises a multi-lumen input port comprising: at least one pressure sensor conduit configured to fluidly couple at an input end with a pressure sensor lumen of a first extraction tube extending from the wound treatment site, and a wound fluid conduit configured to fluidly couple at an input end with a wound fluid lumen of the first extraction tube; and
the output port comprises a multi-lumen output port comprising: an output pressure sensor conduit configured to fluidly couple at an output end with a pressure sensor lumen of a second extraction tube extending to a therapy control unit, and an output wound fluid conduit configured to fluidly couple at an output end with a wound fluid lumen of the second extraction tube.

13. The system of claim 12, wherein each of the plurality of in-line sampling chambers comprises a first pressure sensor coupler, a second pressure sensor coupler, and a negative pressure sensor conduit extending between and fluidly coupling the first pressure sensor coupler with the second pressure sensor coupler establishing a pressure sensor path between the negative pressure sensor input port and the negative pressure sensor output port along the in-line sampling chamber.

14. The system of claim 11, wherein at least one of the theranostic sampling elements of the plurality of in-line sampling chambers comprises a visual theranostic result indicator configured to be triggered to visually indicate a first condition of the wound fluid.

15. The system of claim 11, wherein the chamber switching system comprises a linear track within which each of the plurality of in-line sampling chambers is movably cooperated and along which the plurality of in-line sampling chambers move to sequentially align and couple with the input port and the output port to receive and expose the theranostic sampling element to the wound fluid.

16. The system of claim 11, wherein the chamber switching system comprises a rotational track cooperated with a housing enabling the plurality of in-line sampling chambers to be rotated relative to the housing in sequentially and fluidly coupling the plurality of sampling chambers with the input port and the output port.

17. A method of implementing a negative pressure wound therapy, comprising:
enabling a wound fluid, transported by a first wound fluid lumen in response to a negative wound therapy pressure applied through the first wound fluid lumen from a wound treatment site, to be directed to a first in-line sampling chamber that is removably and fluidly coupled with the first wound fluid lumen to expose a first theranostic sampling element within the first in-line sampling chamber to the wound fluid;
enabling a switching, through a chamber switching system with which the first in-line sampling chamber and an in-line second sampling chamber are cooperated, from the first in-line sampling chamber to the second in-line sampling chamber decoupling the first in-line sampling chamber from a fluid path of the wound fluid and removably and fluidly coupling the second in-line sampling chamber with the first wound fluid lumen; and
enabling the wound fluid to be directed to the second in-line sampling chamber to expose a second theranostic sampling element within the second in-line sampling chamber to the wound fluid.

18. The method of claim 17, further comprising:
maintaining a pressure sensor path between the wound treatment site and a pressure sensor system through a multi-lumen input port fluidly coupling an input end of a pressure sensor conduit of the input port with a pressure sensor lumen extending from the wound treatment site, and through a multi-lumen output port fluidly coupling an output end of a pressure sensor conduit of the output port with a pressure sensor lumen extending to the pressure sensor system.

19. The method of claim 18, wherein the maintaining the pressure sensor path comprises enabling the pressure sensor path to fluidly extend through the first in-line sampling chamber through a releasable and fluid coupling between an output end of the input pressure sensor conduit of the multi-lumen input port and a first pressure sensor coupler of the first in-line sampling chamber, along a negative pressure sensor conduit of the first in-line sampling chamber extending between and fluidly coupling the first pressure sensor coupler with a second pressure sensor coupler.

20. The method of claim 17, wherein the enabling the wound fluid to be directed to the first in-line sampling chamber to expose the first theranostic sampling element to the wound fluid comprises enabling the exposure of the wound fluid to the first theranostic sampling element to cause a triggering of a visual theranostic result indicator to visually indicate a first condition of the wound fluid.

21. The method of claim 17, further comprising:
detecting a termination of an extended negative pressure cycle of a negative pressure wound therapy protocol;
initiating a sampling phase of the negative pressure wound therapy protocol following the termination of the extended negative pressure cycle, and directing the wound fluid to the first sampling chamber over a duration that is less than a duration of the extended negative pressure cycle; and
initiating a subsequent extended negative pressure cycle of the negative pressure wound treatment protocol.

22. The method of claim 17, further comprising:
controlling a valve, cooperated with the first wound fluid lumen, to expose the first wound fluid lumen to an air source and inducing an increased rate of assent of at least some wound fluid along the first wound fluid lumen and into the first in-line sampling chamber.

23. A method of implementing a negative pressure wound therapy, comprising:
applying a negative wound therapy pressure through a first wound fluid lumen from a wound treatment site;
transporting a wound fluid away from the wound treatment site via the first wound fluid lumen in response to the negative wound therapy pressure;
passing the wound fluid through a first in-line sampling chamber that is removably and fluidly coupled with the first wound fluid lumen to expose a first theranostic sampling element within the first in-line sampling chamber to the wound fluid;
switching, using a chamber switching system coupled with the first in-line sampling chamber and a second in-line sampling chamber, from the first in-line sampling chamber to the second in-line sampling chamber to decouple the first in-line sampling chamber from a fluid path of the wound fluid and removably and fluidly coupling the second in-line sampling chamber with the first wound fluid lumen; and
passing the wound fluid through the second in-line sampling chamber to expose a second theranostic sampling element within the second in-line sampling chamber to the wound fluid.
